(19) United States
(12) Patent Application Publication
(10) Pub. No.: US 2021/0122895 A1
(43) Pub. Date: Apr. 29, 2021

(54) CONDUCTIVE CARBON FIBER-BASED SPONGE

(71) Applicant: CARNEGIE MELLON UNIVERSITY, Pittsburgh, PA (US)

(72) Inventors: Ashwati Krishnan, Pittsburgh, PA (US); Pulkit Grover, Pittsburgh, PA (US); Shawn Kelly, Pittsburgh, PA (US); Ritesh Kumar, Pittsburgh, PA (US); Kalee Rozylowicz, Pittsburgh, PA (US)

(21) Appl. No.: 17/047,832

(22) PCT Filed: Jul. 3, 2019
(86) PCT No.: PCT/US2019/040532

§ 371 Date: Oct. 15, 2020

Related U.S. Application Data
(60) Provisional application No. 62/763,868, filed on Jul. 6, 2018.

(51) Int. Cl. C08J 9/00; C08J 9/28; C08J 9/36; C08K 7/06; C08K 3/04; H01B 1/04

(52) U.S. Cl. CPC C08J 9/0085; A61B 5/27; C08J 9/36; C08K 7/06; C08K 3/04; H01B 1/04; C08J 2201/022; C08J 2205/052; C08J 2207/10; C08J 2375/04; C08J 2301/02; C08J 2383/04; C08K 2201/001; C08K 2201/004; C08K 2201/003; C08J 9/286

(57) ABSTRACT

A carbon fiber-based conductive sponge for low electrode-skin impedance biosignal recordings is described. When the sponge is used with water or saline solution, no gel is required, drastically lowering the setup time for EEGs compared to classical wet electrodes. The wet sponges achieve an electrode-skin impedance as low as 2.5 kΩ¶ when wet, making them better than state of the art gel electrodes. Additionally, even as the sponge dries, it continues to remain conductive and performs as a reliable dry electrode.

### RELATED APPLICATIONS

This application claims benefit of US Provisional Patent Application No. 62/763,868, filed Jul. 6. 2018 entitled “Carbon Fiber-Based Conductive Sponge for Electrode-Skin Bio-Potential Measurements”, the contents of which are incorporated herein in their entirety.

### BACKGROUND OF THE INVENTION

The advent of microelectronics has increased our ability to measure and affect the electrical nature of the human both Non-invasive electrical measurements such as electrocardiography (ECG, heart), electroencephalography (EEG, brain) and electromyography (EMG, muscle) etc. are some of the first and the most critical tools in diagnosing and tracking many disorders. For example. EEG is a non-invasive method of measuring the brain's electrical activity used widely in epilepsy diagnosis, studying neurological disorders, neuroscientific studies, and brain-machine interfaces. There have been recent advancements in improving spatial resolution of EEG by increasing the number of sensors. High-Density EEG (HDEEG) systems, using several hundred electrodes, have the potential to become a low-cost imaging technology, but their development is not without challenges. A high-density EEG is illustrated in FIG. 1.

The medium of communication within the body is neuronal electrical signals. Because the dominant medium in the body is aqueous, electrical signals are realized through the movement of ions, as opposed to electrons. When an electrode is placed on the skin for measurement, there is a separation of charge that occurs at the electrode-skin interface. This is because, unlike in the body, electrical current in the electrode amplifier circuit is through the movement of electrons.

Human skin consists of several layers, the outermost of which is the stratum corneum, which acts as a barrier to the flow of ions, thereby increasing the impedance of any electrode material that is placed to acquire signals from the body. To improve SNR, electrode-skin interface impedance needs to be lowered. The skin is inherently a moist material, so technicians obtain the most reliable signals from wet electrodes, which use an electrolyte gel between the electrode and the skin. Wet electrodes provide high signal-to-noise ratio (SNR) but are cumbersome to setup. Dry electrodes have a poor SNR and require a dedicated amplifier to improve the signal.

Although the use of wet electrodes is widespread, they present several problems, especially for HD-EEG. (i) they require the use of special gels that dry out within just a few hours of use; (ii) they take a long time to set up, typically 30-45 minutes for 64 or 128 electrodes; and (iii) the gels tend to spread and cause bridging between adjacent electrodes, thereby reducing the spatial resolution of HD-EEG.

To address these issues, there has been significant progress in use of hydrogels. Hydrogels are materials that retain a large amount of water compared to the material's own volume. They have been incorporated increasingly in commercial disposable EEG electrodes and are a very promising development for EEG. However, hydrogels are unsuitable for long-term use because they lose their conductivity once they dry out. To avoid the use of electrolyte gels, advancements have been made in the design of dry electrodes and sponges.

Portable consumer devices often use dry electrodes that have conductive tips that are directly pushed against the skin, but these offer signals with lower SNR than wet electrodes because of their high impedance. The main idea behind the use of sponges is to use a simple mechanism to “wet” the electrode, by soaking it in an easily available conductive electrolyte, such as a saline solution. The sponge approach is attractive because it is low cost and can be quickly applied. However, the saline solution dries out quickly, and, consequently, the dry sponges are non-conducting. All of the above-mentioned issues become unmanageable for high electrode count HD-EEG systems, and they make long term, ambulatory EEG measurement systems almost impossible.

### SUMMARY OF THE INVENTION

To develop a an biopotential measurement system that is robust, low-cost, and portable, a novel conductive carbon fiber-based conductive sponge is introduced herein that can be used as an electrode for EEG and other applications. The sponge can be easily and frequently re-hydrated for long-term high-quality observations.

When wet electrodes dry out over prolonged use, the electrode-skin impedance can increase to unacceptably high values. A key aspect of the sponges of the present invention is to ensure a low electrode-skin interface impedance, regardless of the wetness of the interface. To that end, a novel foam/sponge that is embedded with conductive carbon fibers is described. When the conductive sponge is infused with saline, it provides an aqueous conductive medium between the electrode rind the skin. Furthermore, due to the presence of conductive carbon fibers, the sponge conducts even when it is dry.

Carbon fibers are strands of carbon having a diameter of ≈5 μm and are mainly carbon atoms bonded together in microscopic crystals. The crystalline arrangement accounts for their high tensile strength. Because carbon fibers comprise mostly carbon (or graphite), they are also good conductors of electricity and are inert to chemical reactions such as corrosion.

In certain embodiments of the invention, the sponge is composed of silicone, cellulose or a hydrophilic polyurethane foam. Silicones are inert, synthetic polymers that have repeating units of siloxanes (Si-O). Silicones are biocompatible, non-corrosive, thermally stable and have been used in the medical field for implants and bandages. These properties make silicone and carbon fibers appealing for their use in portable HDEEG systems. The conductive carbon fiber-based sponge described herein is designed to function as a reliable wet electrode and a convenient dry electrode.

### BRIEF DESCRIPTION OF THE DRAWINGS

FIG. 1 is a schematic drawing of a high-density EEG in situ on a human subject.

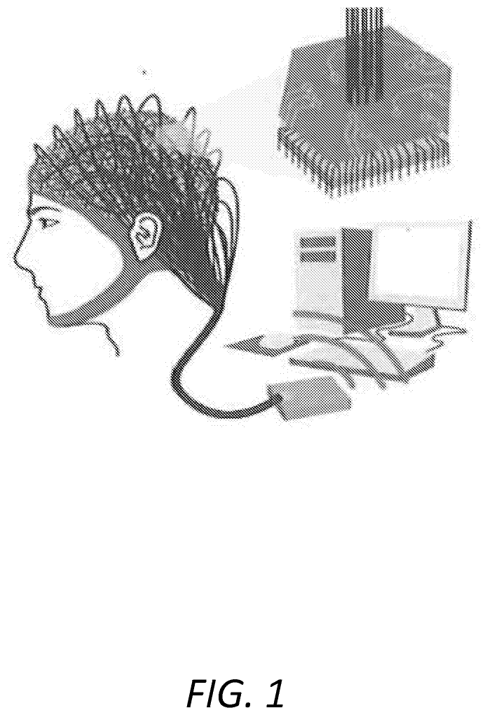

FIG. 2 is drawing of a rectangle or more designed for measuring both conductivity of the conductive sponge.

FIG. 3 is a graph showing conductivity data for carbon fiber silicone sponge samples having various concentrations of carbon fiber content by weight.

FIG. 4 is s a graph showing the rate of evaporation of de-ionized water for carbon fiber silicone sponge samples having various concentrations of carbon fiber content by weight.

FIG. 5 are graphs showing electrode-skin impedance for a conductive sponge as specified herein when dipped in a 0.9% weight by volume saline solution versus other types of electrodes.

FIG. 6 are transient plots from an EEG showing eye blinks illustrating that the conductive sponge of the present invention, when dry, is effective in detecting electrical activity associated with muscle movement on a par even with standard electrodes

FIG. 7 are graphs showing the frequency response of EEG signals acquired from subjects having eyes-open versus eyes-closed, showing that the conductive sponge of the present invention as effective as conventional electrodes.

### DETAILED DESCRIPTION OF THE INVENTION

### Preparation of the Conductive Sponge

In certain embodiments of the invention, a two-part curable silicone foam was used as the sponge medium. Such foam can be obtained, for example, from Smooth-On Inc. of Macungie, Pa. USA, having a brand name of “Soma Foama 15”. Alternatively, hydrophilic pre-polymers from Carpenter Chemicals of Richmond, Va. USA, can be used, which can be cured upon the addition of water. The carbon fiber (CF) may be obtained, for example, from ACP Composites of Livermore. Calif. USA, and typically, a majority of the carbon fibers should be 2-5 mm in length. Alternatively, carbon nanofibers (CNF) can also be used (for example, procured from Pyrograf-III Carbon Nanofiber, Cedarville. Ohio. USA). A majority the Carbon nanofibers should have a diameter of 70-200 nm and a length of 50-200 microns

The silicone foam comes as a two-part preparation, having a Part A being the silicone foam and a Part B being a curing agent. Part A of the two-part silicone foam is thoroughly mixed with the CF at 25° C. in the ratios presented in Table 1 to create a homogenous mixture. Silicone thinning fluid sourced from Hager Plastics of Chicago. Ill. USA, may be added to allow for better flow of the mixture for molding. For the hydrophilic polyurethane, the pre-polymer requires a surfactant that binds with the isocyanate in the polymer to make it more water absorbent. Lauramine oxide and or propylene glycol, a surfactant commonly found in soaps, can be added to the pre polymer before curing. The carbon nanofibers are added thoroughly mixed with the pre polymer before the addition of water.

After thorough mixing, Part B of the silicone foam was added to the Part A-CF blend, stirred and immediately poured into molds to cure. The time taken for the mixture to become a solid foam (cure time) is 1 hour at room temperature. Table 1 shows variations in preparations in different samples for silicone. For the hydrophilic polyurethane sponge, water is added to the pre-polymer-CNF-surfactant mixture and immediately poured into a mold for curing. The time take for curing is about 1 hour at room temperature. Table 2 shows variations for different samples of polyurethane.

TABLE 1
Silicone formulations

| | Silicone (g) | | | Carbon Fiber |
|---|---|---|---|---|
| # | Part A | Part B | Thinning Fluid | (g) |
| I | 3.11 | 1.5 | 0.7 | 0.2 |
| II | 3.07 | 1.5 | 0.62 | 0.25 |
| III | 4 | 2 | 0.2 | 0.6 |
| IV | 4.06 | 2.1 | 0.7 | 0.81 |

TABLE 2
Hydrophilic Polyurethane formulations

Hydrophlic Polyurethane

| | Prepolymer | Surfactant | Water | CNF |
|---|---|---|---|---|
| I | 3 | 0.5 | 4.5 | 1 |
| II | 4.5 | 0.75 | 9 | 1 |
| III | 4.3 | 0.9 | 4.3 | 0.9 |

### Foam Preparation

Foams can be open-cell or closed-cell. Open-cell foams have many interconnected pores, which retain fluid to create an aqueous electrode environment that is required for low electrode-skin impedance. However, most silicone foams are closed-cell foams.

Soma Foama 15 is a closed-cell silicone foam that expands to 4 times its volume through the release of gas bubbles, creating pores. Interior pores can be opened up by applying pressure to the cured foam, or hydrophilic polymers can be used so that the sponge is absorbent.

In alternate embodiments of the invention, different materials may be used for the sponge medium. Any hydrophilic material should be suitable for use as a sponge material. For purposes of use as an EEG electrode, it is preferable that the material be bio-compatible. Preferably, the hydrophilic material starts in liquid form such that the carbon fibers can be mixed in to create a homogenous mixture of the sponge material and the carbon fibers. Thereafter, the sponge material may be solidified in any required way, such as by drying, heating or curing. In certain embodiments of the invention, the hydrophilic material may be a hydrophilic polyurethane foam (described) or a cellulose sponge. In these embodiments, a surfactant may be used to make the polyurethane foam or cellulose more hydrophilic.

The carbon fiber needs to be mixed until the Pan A-CF blend appears homogeneous (in the case of Soma Foama 15 with a shiny grey texture). This is because conduction in the silicone occurs through interconnected fibers that separate while mixing Graphite powder or milled carbon fiber was not as effective in increasing the conductivity of the silicone foam. Once the sample has cured, about 1 mm of all surfaces needed to be cut or filed to expose these fibers to metal contacts.

Chopped carbon fibers of length ˜6 mm are commercially available. However, this length makes the silicone-CF mixture difficult to pour into molds because it behaves like a flat sheet, rather than a pourable mixture. The pot life (the time elapsed before the mixture starts to cure) of Soma Foama 15 is 30 seconds. Thus, it needs to be poured immediately after mixing in Part B, and this can be accomplished more reliably with shorter carbon fibers or carbon nano-fibers.

The CF changes the mechanical properties of the resulting foam. If too much CF is added, the resulting mixture is too heavy to expand into a foam with many pores. In such cases, CNF max prove to be more reliable. There is a trade-off between foam expansion and electrical conduction.

### Material Properties

The material characteristics shown here are relevant to EEG recordings. Table 1 shows a comparison of the conductivity of the CF sponge, and the extent of water retention for various mixture ratios.

### Conductivity

The conductivity of bulk materials is obtained by measuring the resistance of a sample of known geometry by forcing a current through one pair of leads and measuring the voltage through another pair. 3D printed rectangular molds were used to study the conductivity of the CF sponge. The conductivity was measured using a Keithley 2400 source-meter (Tektronix, Inc., Beaverton, Oreg. USA) and was measured when the CF sponge was dry as well as after absorbing 0.9% w/v saline solution, which has a conductivity of 14.7 milli-Siemens per centimeter.

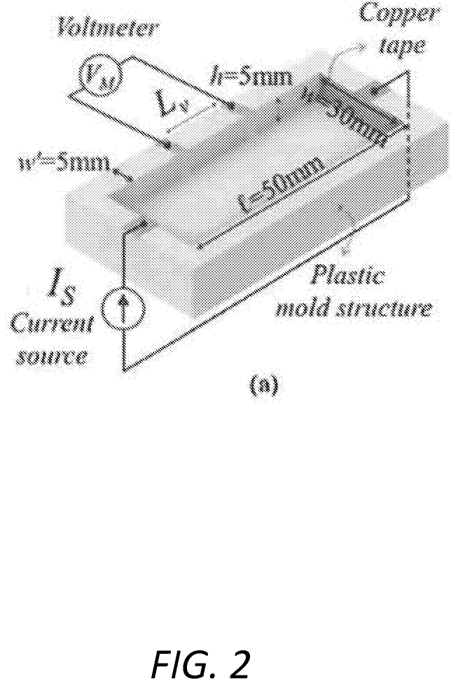

FIG. 2 shows the dimensions of the mold and the circuit configuration used to perform the tests. The conductivity, σ, of the bulk material is given by:

$$σ = \frac{{I}_{S}}{{V}_{M}} * \frac{{L}_{v}}{w * h}$$

where the variable notations are provided in FIG. 2.

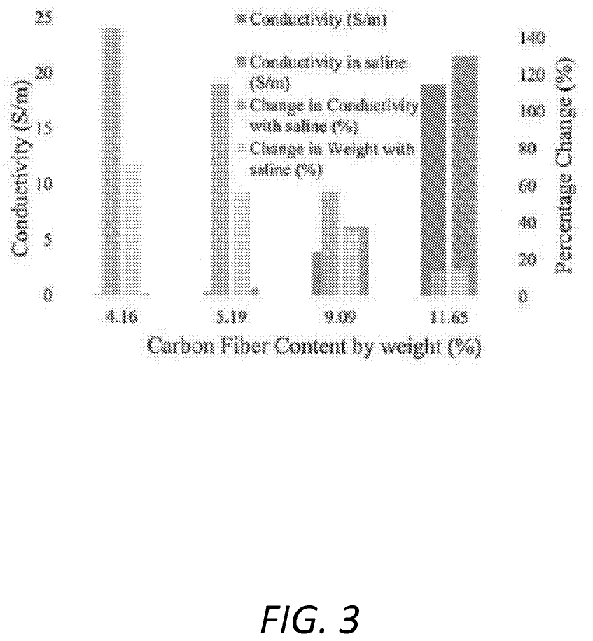

The results of the tests are shown in FIG. 3. Using the 4-point measurement technique for bulk materials, the conductivity of the carbon fiber-based sponges was shown to vary with the amount of CF in the silicone sponge. The conductivity of the sample increases with CF and in the presence of saline. The change in conductivity due to the addition of saline decreases with increase in CF, because higher concentration of CF implies fewer pores in the material to hold in the saline solution. The sponge structure ensures the presence of an aqueous ionic solution for a low electrode-skin impedance. Similar plots may be obtained for the hydrophilic poly urethane sponge.

### Water Retention

The samples shown in Table 1 were squeezed in de-ionized water, dabbed on a clean paper towel to remove the excess drip and placed in a standard temperature and pressure environment. The samples were weighed repeatedly over 10 hours to observe the extent of evaporation over time. Similar plots may be obtained for the hydrophilic polyurethane sponge formulations in Table 2.

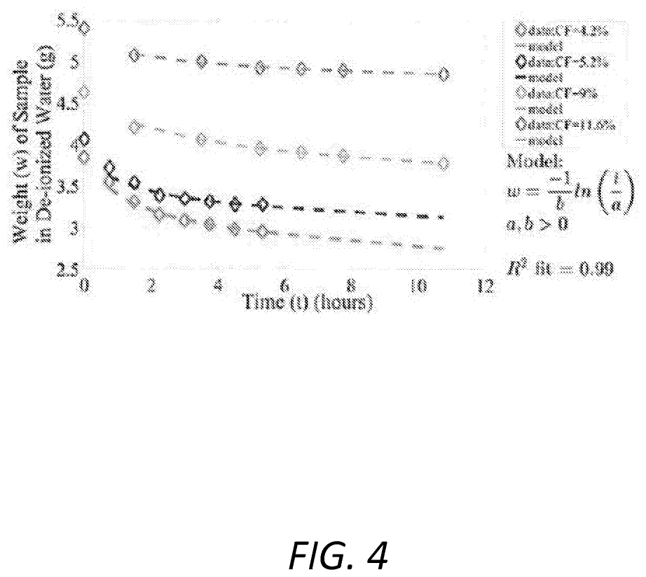

To evaluate the extent of liquid retention, the rate of evaporation of de-ionized water in a few silicone samples over several hours was measured, and the results are shown in FIG. 4. The results show that the weight of the sample undergoing evaporation decreased in a logarithmic manner. The data are shown in FIG. 4 along with the generalized model equation.

### Human Scalp Measurements

To evaluate the efficacy of the conductive carbon fiber silicone sponge electrodes for biosignal acquisition applications, impedance measurements and EEG recordings on a human participant were performed. Electrode-skin impedance measurements were performed using the Intan Recording Controller (Los Angeles, Calif., USA) A sampling rate of 20 kilosamples/sec, bandpass filter settings of 0.1 Hz to 7.5 kHz and a notch filter setting at 60 Hz were used. Conductive sponge electrodes in wet and dry conditions were compared to a Covidien Kendall (Minneapolis, Minn. USA) disposable hydrogel electrode, a BrainVision (M01Tisville, N.C. USA) fiat, metal passive dry electrode and a gold-cup electrode (Natus Neurology, Pleasanton, Calif. USA) (FIG. 2d).

The diameter of all electrodes was between 8-10 mm and the thickness of the conductive carbon fiber-based sponge electrodes was 2-4 mm. For these experiments, one electrode of each of the 4 types was placed close together on the left and right sides of the forehead.

### Electrode-Skin Impedance

While electrode impedance values are typically reported at 1 kHz, many relevant EEG signals are at a much lower frequency (5-40 Hz). Therefore, electrode-skin impedance was recorded at values at 20 Hz, 200 Hz, 1 kHz and 3 kHz.

The akin was not abraded for the electrodes under evaluation, however, a gold-plated cup electrode with Ten20 conductive paste was placed over abraded skin on the right mastoid bone as a reference to ensure an unbiased comparison. To verify the low impedance of the reference, an identical cup electrode configuration over the left mastoid was also used.

### EEG Measurements

Alpha waves am a highly stereotypical form of EEG activity that can be measured when the participant is in a relaxed state, or when their eyes are closed 3 minutes of EEG signals from a participant were measured under two conditions: with eyes open and eyes closed. A frequency analysis of the acquired data was performed using a MATLAB-based EEGLAB toolbox.

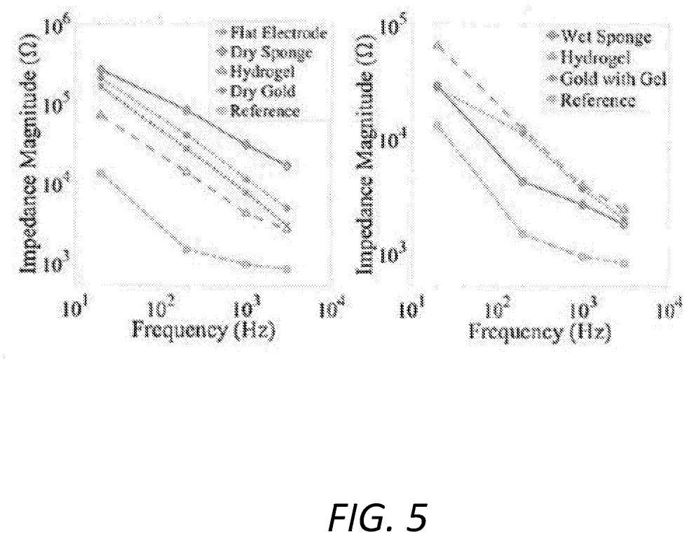

The magnitude of the electrode-skin impedance is shown in FIG. 5. The reference electrode impedance was between 0.3-0.5 kΩ. The wet conductive sponge electrode achieved an impedance of around 2 kΩ, which was lower than the wet gold cup electrode with Signa electrode gel and the disposable hydrogel electrode. The impedance of the dry CF-sponge electrode was comparable to that of standard dry electrodes.

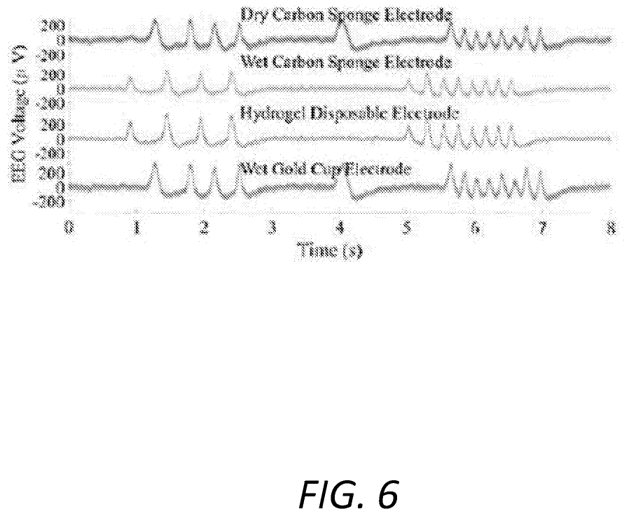

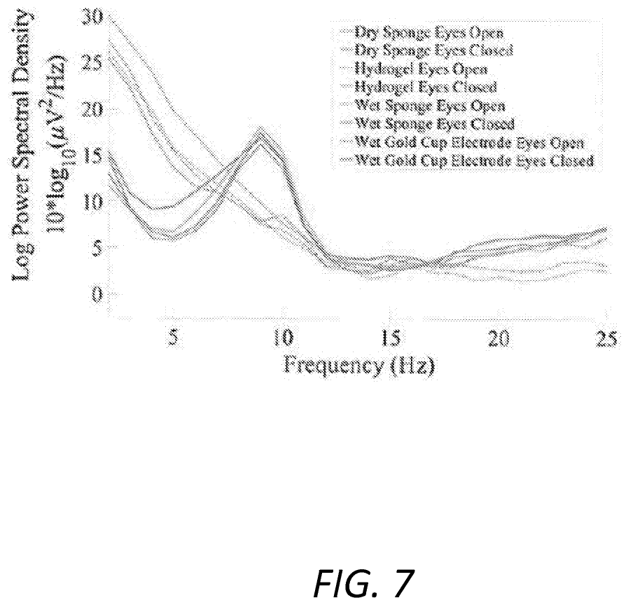

To demonstrate the efficacy of the conductive carbon fiber electrode material as an electrode to detect muscular activity, a time series plot is shown in FIG. 6, depicting different rates of blinking. Alpha wave measurements manifest when people close their eyes and are typically within 8-12 Hz. FIG. 7 shows the frequency spectrum peaking in the presence of alpha waves when eyes are closed and absent when eyes are open. While it has been well established that wet electrodes are a reliable means detecting alpha waves, the dry conductive sponge electrodes are as effective as wet electrodes in measuring alpha wave activity in the brain.

A novel carbon liber-based conductive sponge for use in biomedical applications such as EEG has been described herein. As the percentage of carbon fiber in the sponge increases, the conductivity also increases. On the other hand, the amount of solution the material can hold decreases, because there are fewer pores in the material.

A lower electrode-skin impedance was observed with a dry conductive sponge with high carbon fiber content (9-11 %). Increasing fiber content reduces the amount of time the electrode can be used as a wet electrode. The impedance of two 9 mm diameter circular carbon fiber-based sponges soaked in 0.9% w/v saline solution was tin average of 2.5 kΩ, which is better than a gold electrode with electrolyte gel. The conductive sponge electrodes (dry and wet) can reliably measure alpha waves on the forehead.

The conductive carbon-fiber sponge electrodes are a low cost, fast-installation solution for high-quality biosignal measurements. They are non-magnetic, so they can be used in conjunction with Magnetic Resonance Imaging (MRI) machines.

Because there is no electrode gel involved, the delivery of saline solution is a convenient way to achieve excellent wet electrodes within a short setup time. The purpose of using a conductive sponge is to maintain a low electrode-skin impedance even as the electrode dries out. The carbon fiber-based conductive sponge electrodes have particular applicability in portable ambulatory and low-cost high density biosignal measurement systems.

We claim:

1. A conductive sponge comprising:
a sponge body comprising a hydrophilic material; and
a plurality of carbon fibers or carbon nanofibers dispersed throughout the sponge body.

2. The conductive sponge of claim 1 wherein the hydrophilic material is hydrophilic polyurethane foam or cellulose.

3. The conductive sponge of claim 2 further comprising:
adding a surfactant to the hydrophilic material.

4. A conductive sponge comprising:
a sponge body comprising a silicone foam: and
a plurality of carbon fibers dispersed throughout the sponge body.

5. The conductive sponge of claim 4 wherein the plurality of carbon fibers comprises between 5% and 12% of the total weight of the conductive sponge.

6. The conductive sponge of claim 4 wherein the silicone foam is a closed-cell foam.

7. The conductive sponge of claim 4 wherein the carbon fibers range in length from approximately 2 mm to approximately 5 mm.

8. The conductive sponge of claim 4 wherein a majority of the carbon fibers are between 2 mm and 5 mm in length.

9. The conductive sponge of claim 4 wherein the carbon fibers are approximately 5 microns in diameter.

10. The conductive sponge of claim 4 wherein the conductive sponge is conductive when dry.

11. A conductive sponge comprising:
a sponge body comprising a hydrophilic material or a silicone foam; and
a plurality of carbon nanofibers dispersed throughout the sponge body.

12. A process for manufacturing a conductive sponge comprising:
mixing a plurality of carbon fibers or carbon nanofibers into an uncured silicon foam or a hydrophilic polyurethane pre-polymer to create a homogenous mixture;
mixing a curing agent to the homogeneous mixture of uncured silicone foam and carbon fibers, or mixing surfactant and water with the homogenous polyurethane mixture; and
pouring the mixture into a mold for curing.

13. The process of claim 12 further comprising adding a thinning agent to the homogenous silicone mixture prior to the pouring of the mixture into a mold.

14. The process of claim 12 further comprising shaving a layer from one or more surfaces of the cured conductive sponge to expose the carbon fibers.

15. The process of claim 12 wherein a majority of the carbon fibers are between 2 mm and 5 mm in length, or wherein the majority of the carbon nanofibers are 50-200 microns in length.

16. The process of claim 12 wherein the carbon fibers are approximately 5 microns in diameter, or carbon nanofibers are approximately 0.1 microns in diameter

17. The process of claim 12 wherein the carbon fibers comprise between 5% and 12% of the total weight of the conductive sponge.
